(19) United States
(12) Patent Application Publication
(10) Pub. No.: US 2021/0244403 A1
(43) Pub. Date: Aug. 12, 2021

(54) SYSTEM AND METHOD FOR ATTACHING A PRE-SUTURED TENDON TO AN ADJUSTABLE LOOP FIXATION DEVICE

(71) Applicant: Conmed Corporation, Largo, FL (US)

(72) Inventor: Giuseppe Lombardo, New Port Richey, FL (US)

(73) Assignee: Conmed Corporation, Largo, FL (US)

(21) Appl. No.: 17/172,394

(22) Filed: Feb. 10, 2021

Related U.S. Application Data
(60) Provisional application No. 62/972,322, filed on Feb. 10, 2020.
(60) Provisional application No. 62/972,332, filed on Feb. 10, 2020.

(51) Int. Cl. A61B 17/04

(52) U.S. Cl. CPC A61B 17/0401; A61B 2017/0412; A61B 2017/0404

(57) ABSTRACT

A system and method of repairing an ACL using a fixation device and a pre-sutured tendon. The fixation device includes a button having first and second inner apertures extending therethrough and a length of suture having a first limb connected to a second limb. The first and second limbs both extend through the first inner aperture, forming an adjustable loop in the length of suture extending from the button. A mass of suture material or a protuberance is formed in a first terminal end of the first limb extending proximally from the button. The mass of suture material and the protuberance have a diameter that is larger than a diameter of the first inner aperture. Pulling the second limb proximally from the button decreases the size of the adjustable loop.

### CROSS-REFERENCE TO RELATED APPLICATION

The present application claims priority to and the benefit of U.S. Provisional Patent Application No. 62/972,322, filed on Feb. 10, 2020 and entitled “System and Method for Attaching a Pre-Sutured Tendon to an Adjustable Loop Fixation Device,” and U.S. Provisional Patent Application No. 62/972,332, filed on Feb. 10, 2020 and entitled “System and Method for Attaching a Pres-Suture Tendon to an Adjustable Loop Fixation Device,” the entireties of which are incorporated herein by reference.

### BACKGROUND OF THE INVENTION

### 1. Field of the Invention

The present invention relates to a surgical fixation construct and, more particularly, to a system and method of repairing an ACL using a fixation device and a pre-sutured tendon.

### 2. Description of Related Art

Using a pre-sutured tendon is advantageous to both the patient and surgeon. The patient avoids the post-op morbidity associated with taking an autograft. This elimination of harvesting the graft results in shorter OR time, resulting in reduced risk to both the patient and surgeon. A pre-sutured tendon consists of a viable allograft tissue that is sutured at both ends creating an oval closed loop. If the surgeon wishes to use suspensory fixation, the surgeon must attach such a device to either or both ends of the pre-sutured tendon. Many suspensory fixation devices have a continuous closed loop or an adjustable closed loop. These options are not ideal for attaching to a pre-sutured tendon. Therefore, a need exists for an adjustable loop suspensory device where the adjustable loop can be formed around the pre-sutured tendon.

The term “suture” as used herein may be any type of filamentous material such as a biocompatible or bioabsorbable filament, ribbon, tape, woven or non-woven material.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY OF THE INVENTION

Embodiments of the present invention are directed to a system and method of repairing an ACL using a fixation device and a pre-sutured tendon. The fixation device includes a button having first and second inner apertures extending therethrough and a length of suture having a first limb connected to a second limb. The first and second limbs both extend through the first inner aperture, forming an adjustable loop in the length of suture extending from the button. A mass of suture material or a protuberance is formed in a first terminal end of the first limb extending proximally from the button. The mass of suture material and the protuberance have a diameter that is larger than a diameter of the first inner aperture. Pulling the second limb proximally from the button decreases the size of the adjustable loop.

According to another aspect, the present invention is a fixation construct. The fixation construct includes a button having first and second inner apertures extending therethrough and a length of suture having a first limb connected to a second limb. The first and second limbs both extend through the first inner aperture, forming an adjustable loop in the length of suture extending from the button. A mass of suture material or a protuberance is formed in a first terminal end of the first limb extending proximally from the button. The mass of suture material and the protuberance have a diameter that is larger than a diameter of the first inner aperture. Pulling the second limb proximally from the button decreases the size of the adjustable loop. The fixation construct also includes a tendon connected to the button by the adjustable loop such that the adjustable loop extends through the tendon and the second inner aperture and is secured on a proximal side of the button.

According to another aspect, the present invention is a method for creating a fixation construct. The method includes: (i) providing a fixation device having a button having first and second inner apertures extending therethrough, a length of suture having a first limb connected to a second limb, the first and second limbs both extending through the first inner aperture, forming an adjustable loop in the length of suture extending from the button, a mass of suture material or a protuberance formed in a first terminal end of the first limb extending proximally from the button, wherein the mass of suture material and the protuberance have a diameter that is larger than a diameter of the first inner aperture; (ii) attaching a passing strand to the adjustable loop, the passing strand having a free end; (iii) passing the free end of the passing strand through a tendon; (iv) extending the free end of the passing strand through the second inner aperture until the adjustable loop is passed through the second inner aperture and extends from a proximal side of the button; (v) securing the adjustable loop on the proximal side of the button; and (vi) pulling the second limb proximally from the button.

These and other aspects of the invention will be apparent from and elucidated with reference to the embodiment(s) described hereinafter.

### BRIEF DESCRIPTION OF THE SEVERAL VIEWS OF THE DRAWING(S)

The present invention will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1A is a perspective view of a fixation device, according to an embodiment;

FIG. 1B is a perspective view of detail A of the fixation device of FIG. 1A;

FIG. 2 shows a perspective view, diagrammatic representation for a method for creating a mass of suture material, according to an embodiment;

FIG. 3 shows a perspective view of the fixation device connected to a tendon in a pre-deployment configuration, according to an embodiment;

FIG. 4A shows a perspective view of the fixation device connected to the tendon in a first deployment configuration, according to an embodiment;

FIG. 4B shows a perspective view of detail B of the fixation device of FIG. 4A;

FIG. 5A shows a perspective view of the fixation device connected to the tendon in a second deployment configuration, according to an embodiment;

FIG. 5B shows a perspective view of detail C of the fixation device of FIG. 5A;

FIG. 6A shows a perspective view of the fixation device connected to the tendon in a post-deployment configuration, according to an embodiment;

FIG. 6B shows a perspective view of detail D of the fixation device of FIG. 6A;

FIG. 7A shows a perspective view of a fixation device, according to an alternative embodiment;

FIG. 7B shows a perspective view of detail A of the fixation device of FIG. 7A;

FIG. 8 shows a perspective view of the fixation device connected to a tendon in a pre-deployment configuration, according to an alternative embodiment;

FIG. 9 shows a perspective view of the fixation device connected to the tendon in a first deployment configuration, according to an alternative embodiment;

FIG. 10A shows a perspective view of the fixation device connected to the tendon in a second deployment configuration, according to an alternative embodiment;

FIG. 10B shows a perspective view of detail C of the fixation device of FIG. 10A;

FIG. 11A shows a perspective view of the fixation device connected to the tendon in a post-deployment configuration, according to an alternative embodiment; and

FIG. 11B shows a perspective view of detail D of the fixation device of FIG. 11B.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

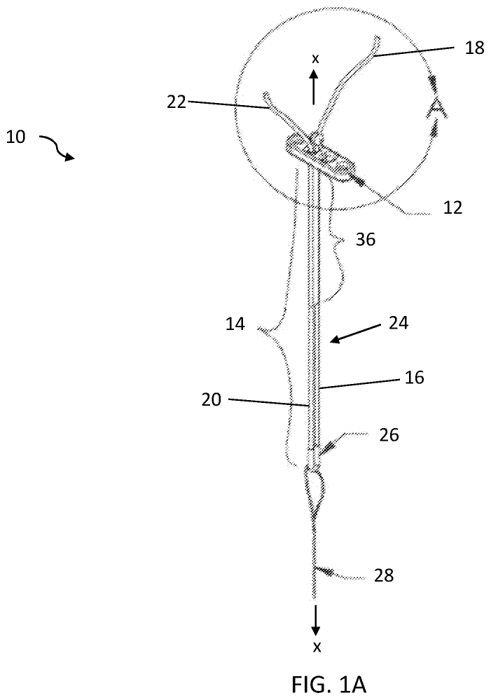

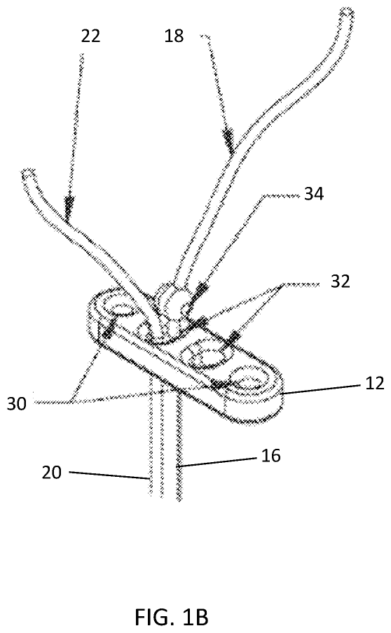

Referring now to the figures, wherein like reference numerals refer to like parts throughout, FIG. 1A shows a perspective view of a fixation device 10, according to an embodiment. The fixation device 10 comprises a button 12 with a length of suture 14 extending therethrough. The length of suture 14 comprises a first limb 16 with a first terminal end 18 and a second limb 20 with a second terminal end 22. The first and second limbs 16, 20 extend through the button 12, as described in detail below.

Still referring to FIG. 1A, the length of suture 14 comprises an adjustable loop 24 extending distally from the button 12. The adjustable loop 24 formed in the length of suture 14 comprises a collapsible mechanism 26. The collapsible mechanism 26 in the embodiment of the fixation device 10 in FIG. 1A is a collapsible barb passed over the length of suture 14. The collapsible barb 26 can be composed of a hollow braided tubular structure that is coaxial with the adjustable loop 24 (i.e., first and second limbs 16, 20) or a flat woven structure that has the length of stuture 14 (at the adjustable loop 24) woven through at several locations.

In the embodiment depicted in FIG. 1A, the collapsible barb 26 extends on or connects to both the first limb 16 and the second limb 20. At a point normal to a central longitudinal axis x-x extending through the adjustable loop 24 (between the first and second limbs 16, 20) and the collapsible barb 26, a passing strand 28 extends through and/or connects to the adjustable loop 24. The passing strand 28 is interposed to prevent relative motion between the collapsible barb 26 and the adjustable loop 24 during manipulation.

Turning now to FIG. 1B, there is shown a perspective view of detail A of the fixation device 10 of FIG. 1A. As shown in FIG. 1B, the first and second terminal ends 18, 22 of the first and second limbs 16, 20, respectively, extend proximally from the button 12. The button 12 is oblong and comprises two outer apertures 30 and two inner apertures 32. The two inner apertures 32 are between the two outer apertures 30, as shown. In the depicted embodiment, the first and second terminal ends 18, 22 of the first and second limbs 16, 20, respectively, extend through one of the inner apertures 32. As also shown in FIG. 1B, the first limb 16 comprises a mass 34 of suture material formed at or near the first terminal end 18. The mass 34 of suture material is larger than the inner aperture 32 that the first limb 16 extends through. In other words, the mass 34 of suture material has a diameter that is larger than a diameter of the inner aperture 32.

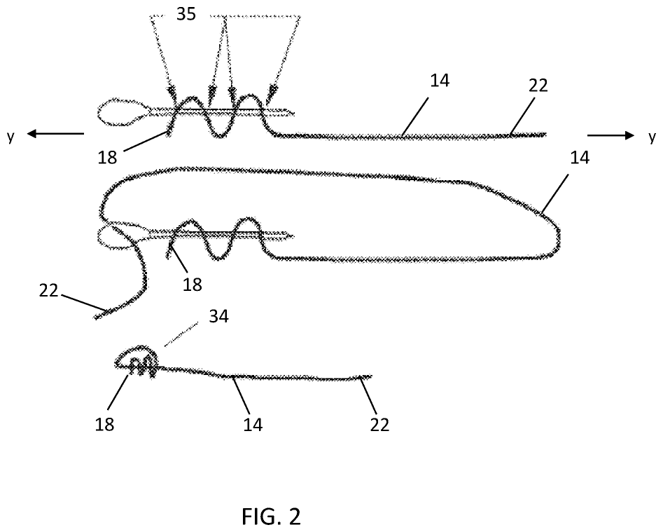

Referring now to FIG. 2, there is shown a perspective view, diagrammatic representation for a method for creating the mass 34 of suture material, according to an embodiment. Prior to loading the length of suture 14 on the button 12 as shown in FIGS. 1A-1B, the mass 34 is created in the length of suture 14. The mass 34 is created by forming multiple passages 35 transversely across the major axis y-y of the length of suture 14 at the first terminal end 18 and then passing the second terminal end 22 through the multiple passages 35 to create the mass 34 of suture material.

Also prior to loading the length of suture 14 on the button 12, in the same first limb 16 comprising the mass 34 of suture material, an eye-splice 36 (FIG. 1A) is created and the opposite, second limb 20 is passed through it, creating the adjustable loop 24 (FIG. 1A) in the fixation device 10. The length of the eye-splice 36 is at least 21 times a diameter of the first limb 16 (or second limb 20). The adjustable loop 24 is disposed between the two inner apertures 32. The outer apertures 30 are used to attach additional limbs of suture (not shown) to manipulate the button 12 into the bone socket and through the cortical aperture as done in ordinary ACL suspensory fixation.

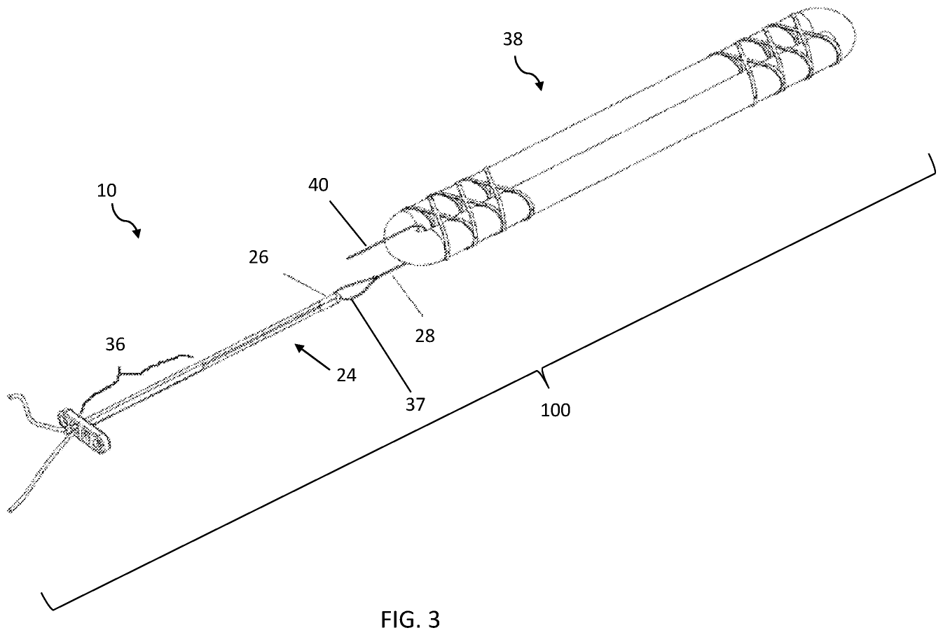

Turning now to FIG. 3, there is shown a perspective view of the fixation device 10 connected to a tendon 38 in a pre-deployment configuration, according to an embodiment. In the pre-deployment configuration, the fixation device 10 is loaded onto a tendon, particularly, a pre-sutured tendon 38 as shown in FIG. 3. This normally occurs on the back table of an operating room. To attach the pre-sutured tendon 38 to the fixation device 10, the passing strand 28 is connected to the adjustable loop 24. As shown in FIG. 3, the passing strand 28 has a closed loop 37 that is passed through the adjustable loop 24 of the fixation device 10. Specifically, the closed loop 37 of the passing strand 28 extends through the adjustable loop 24 against the collapsible barb 26. A free end 40 of the passing strand 28 is passed through the pre-sutured tendon 38 to load the pre-sutured tendon 38 onto the fixation device 10 to create a fixation construct 100.

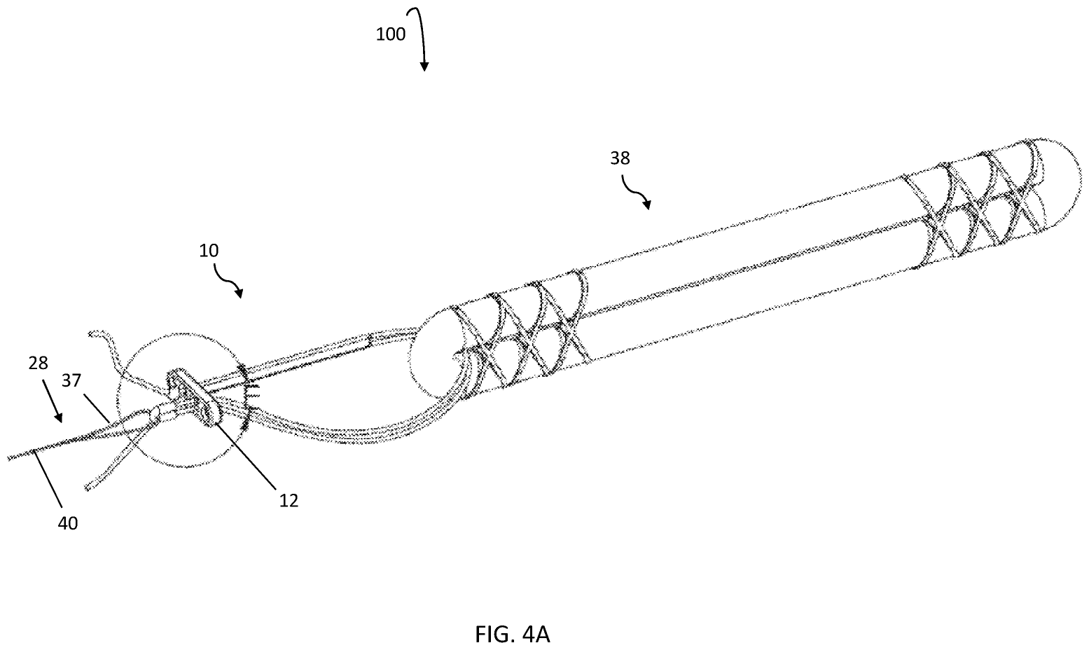

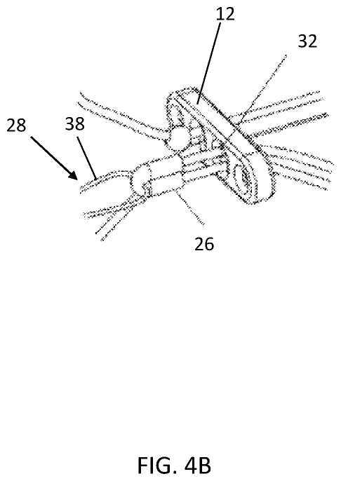

Referring now to FIG. 4A, there is shown a perspective view of the fixation device 10 connected to the tendon 38 in a first deployment configuration, according to an embodiment. Once passed through the pre-sutured tendon 38, the free end 40 of the passing strand 28 is passed through one of the inner apertures 32 of the button 12. The inner aperture 32 is shown in the perspective view of detail B in FIG. 4B. The free end 40 (FIG. 4A) of the passing strand 28 is tensioned or pulled until the collapsible barb 26 also passes through the inner aperture 32. In FIG. 4B, the collapsible barb 26 is shown on a proximal side of the button 12 and extending proximally from the button 12.

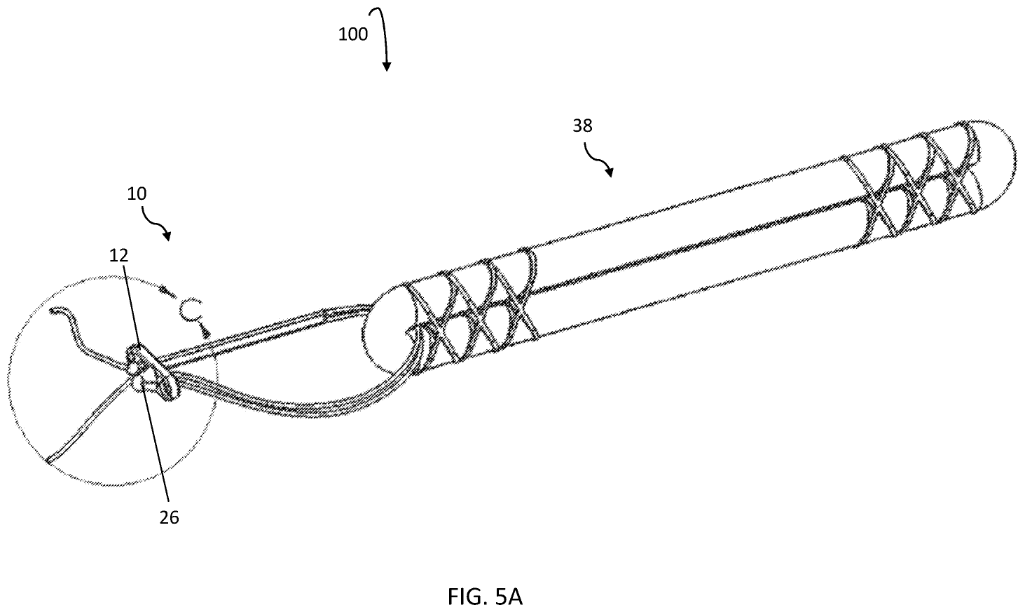

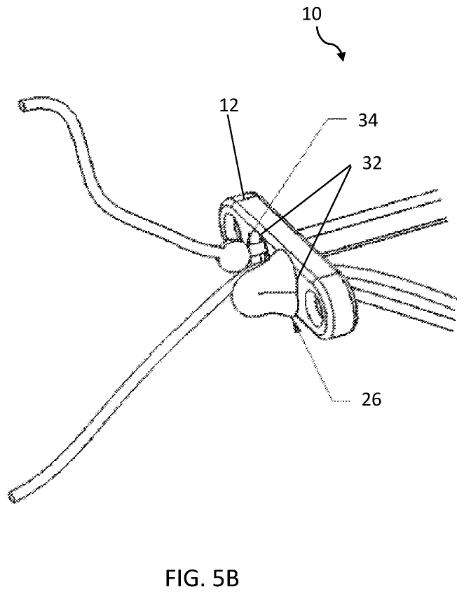

Turning now to FIG. 5A, there is shown a perspective view of the fixation device 10 connected to the tendon 38 in a second deployment configuration, according to an embodiment. Once the collapsible barb 26 is passed through the button 12 and on a proximal side of the button 12, the passing strand 28 (FIG. 4A) is removed from the fixation device 10 and the pre-sutured tendon 38. The collapsible barb 26 is then tensioned or pulled in the distal direction (via pulling the first limb 16 proximally or pulling the tendon 38 distally), which causes the collapsible barb 26 to be pulled retrograde against the button 12 to deploy or collapse the collapsible barb 26 to capture it. As a result, the collapsible barb 26 is compressed against the proximal side of the button 12, causing it to radially expand larger than a diameter of the inner aperture 32, as shown in FIG. 5B.

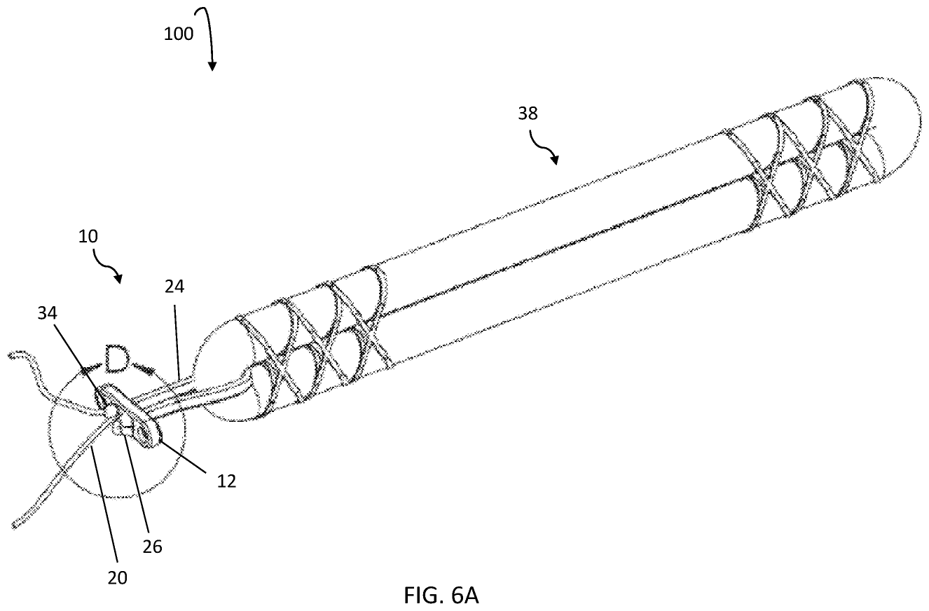

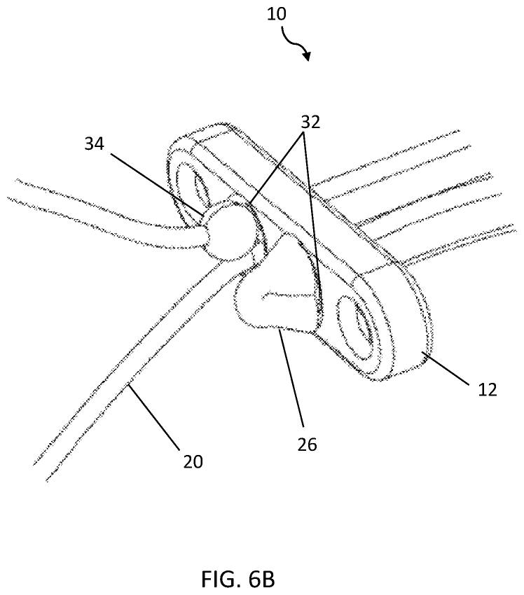

Referring now to FIG. 6A, there is shown a perspective view of the fixation device 10 connected to the tendon 38 in a post-deployment configuration, according to an embodiment. The construct 100 is reduced by pulling the second limb 20. In particular, pulling the second limb 20 reduces the size (i.e., diameter or circumference) of the adjustable loop 24, bringing the pre-sutured tendon 38 toward the button 12. The second limb 20 is pulled until the desired length of the adjustable loop 24 is achieved and the pre-sutured tendon 38 is in the desired location respective to the button 12. Pulling the second limb 20 also further compresses the collapsible barb 26 and brings the mass 34 of suture material to rest against the inner aperture 32 in the button 12 adjacent to the collapsible barb 26, which is against the other inner aperture 32 in the button 12, as shown in FIG. 6B.

Once the fixation device 10 is attached to the pre-sutured tendon 38, forming the fixation construct 100, the fixation construct 100 is passed through into a bone socket which communicated through both the inner and outer cortex. The fixation construct 100 is pulled through the bone socket using the first and second limbs 16, 20 until it emerges on the far cortex and flips on the surface. Details of the graft passing procedure are provided in U.S. Pat. No. 9,931,197, assigned to the assignee hereof and incorporated in its entirety herein by reference.

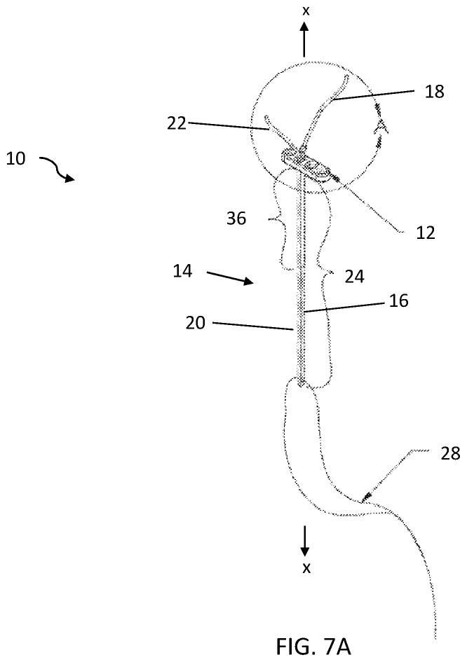

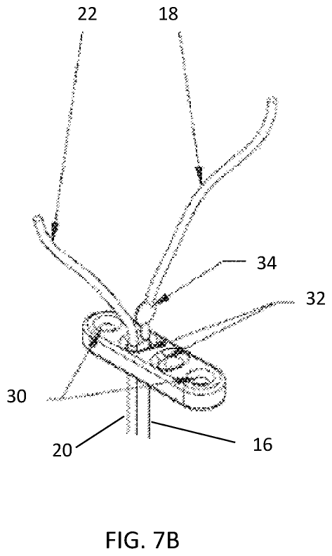

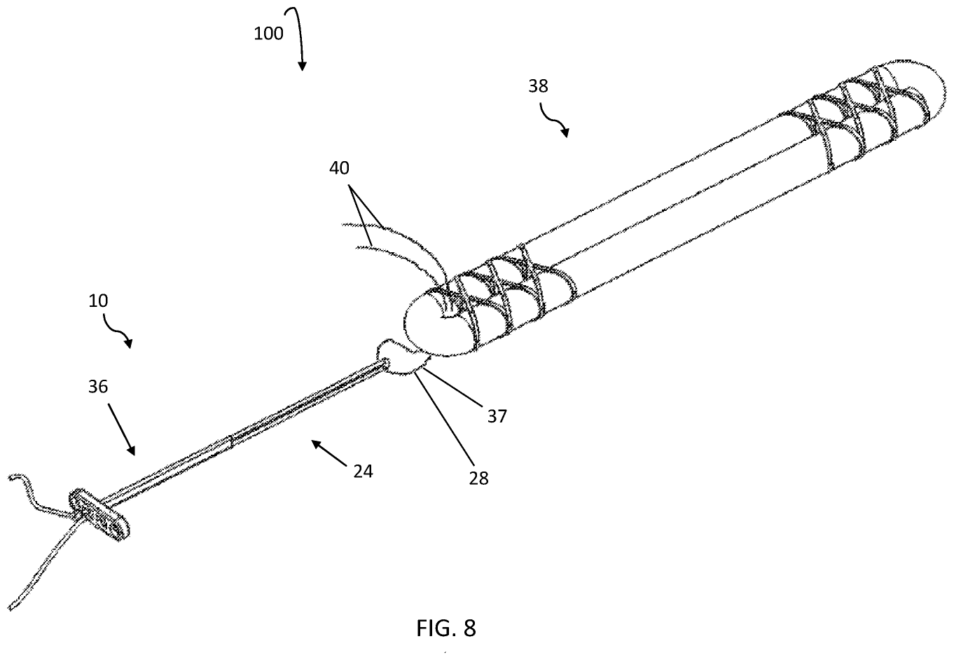

Turning now to FIGS. 7A-11B, there are shown various views of an alternative embodiment of a fixation device 10 for connection to a pre-sutured tendon 38 to create a construct 100 (FIGS. 8-11B). FIG. 7A shows a perspective view of a fixation device 10, according to an alternative embodiment. The fixation device 10 comprises a button 12 with a length of suture 14 extending therethrough. The length of suture 14 comprises a first limb 16 with a first terminal end 18 and a second limb 20 with a second terminal end 22. The first and second limbs 16, 20 extend through the button 12, as described in detail below.

Still referring to FIG. 7A, the length of suture 14 comprises an adjustable loop 24 extending distally from the button 12 formed by the first limb 16 and the second limb 18. At a point normal to a central longitduinal axis x-x extending through the adjustable loop 24 (between the first and second limbs 16, 20), a passing strand 28 extends through and/or connects to the adjustable loop 24.

FIG. 7B shows a perspective view of detail A of the fixation device 10 in FIG. 7A. As shown in FIG. 7B, the first and second terminal ends 18, 22 of the first and second limbs 16, 20, respectively, extend proximally from the button 12. The button 12 is oblong and comprises two outer apertures 30 and two inner apertures 32. The two inner apertures 32 are between the two outer apertures 30, as shown. In the depicted embodiment, the first and second terminal ends 18, 22 of the first and second limbs 16, 20, respectively, extend through one of the inner apertures 32.

As also shown in FIG. 7B, the first limb 16 comprises a protuberance 34 formed at or near the first terminal end 18 extending from a proximal side of the button 12. The protuberance 34 can be created by tying a knot or similar structure in the first limb 16. According to an embodiment, the protuberance 34 is larger than the inner aperture 32 that the first limb 16 extends through. In other words, the protuberance 34 has a diameter that is larger than a diameter of the inner aperture 32.

Referring back to FIG. 7A, prior to loading the length of suture 14 on the button 12, in the same first limb 16 comprising the protuberance 34, an eye-splice 36 is created and the opposite, second limb 20 is passed through it, creating the adjustable loop 24. The length of the eye-splice 36 is at least 21 times a diameter of the first limb 16 (or second limb 20). The adjustable loop 24 is disposed between the two inner apertures 32. The outer apertures 30 are used to attach additional limbs of suture (not shown) to manipulate the button 12 into the bone socket and through the cortical aperture as done in ordinary ACL suspensory fixation.

Turning now to FIG. 8, there is shown a perspective view of the fixation device 10 connected to a tendon 38 in a pre-deployment configuration, according to an alternative embodiment. In the pre-deployment configuration, the fixation device 10 is loaded onto a tendon, particularly, a pre-sutured tendon 38 as shown in FIG. 8, to create the fixation construct 100. This normally occurs on the back table of an operating room. To attach the pre-sutured tendon 38 to the fixation device 10, the passing strand 28 is looped through or otherwise connected to the adjustable loop 24. The passing strand 28 can form a closed loop 37 through the adjustable loop 24. As shown in FIG. 8, the passing strand 28 extends through the adjustable loop 24 such that free ends 40 of the passing strand 28 extends therefrom (or from the closed loop 37). The free ends 40 are passed through the pre-sutured tendon 38, as shown.

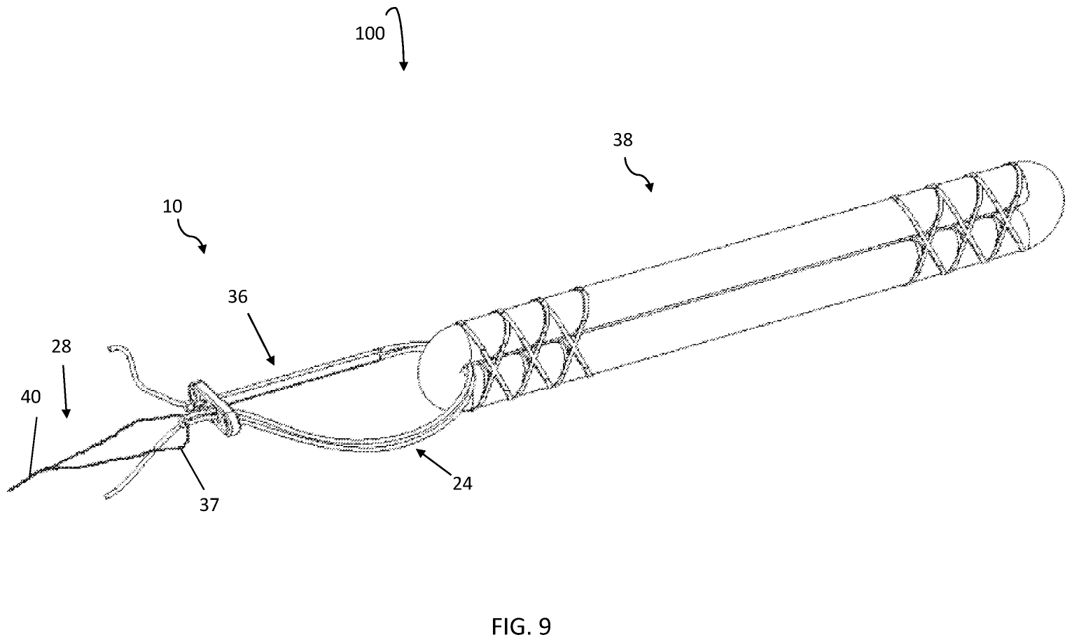

Referring now to FIG. 9, there is shown a perspective view of the fixation device 10 connected to the tendon 38 in a first deployment configuration, according to an alternative embodiment. Once passed through the pre-sutured tendon 38, the free ends 40 of the passing strand 28 are passed through one of the inner apertures 32. In particular, the free ends 40 of the passing strand 28 are passed through an open inner aperture 32, i.e., the inner aperture 32 that does not have the first and second limbs 16, 20 extending therethrough. The free ends 40 of the passing strand 28 are tensioned or pulled until the adjustable loop 24 also passes through the inner aperture 32.

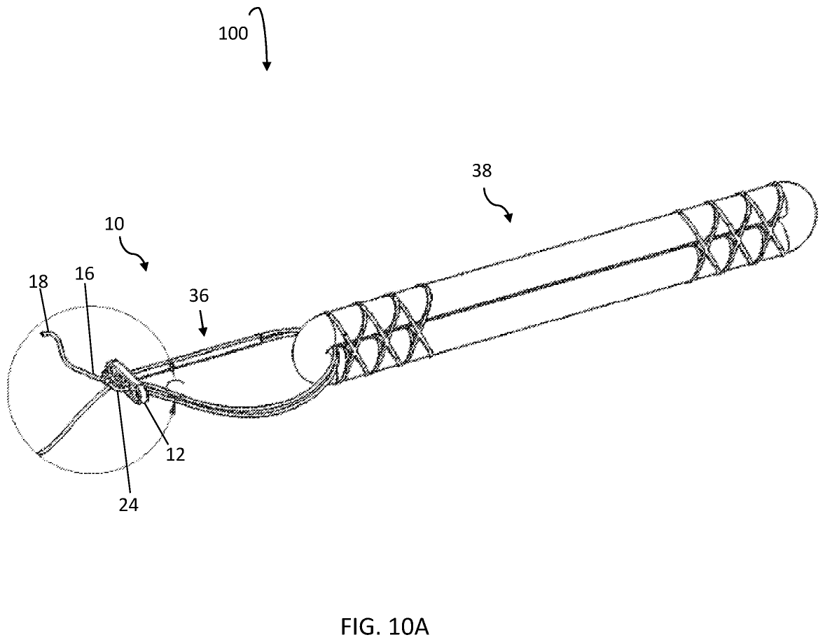

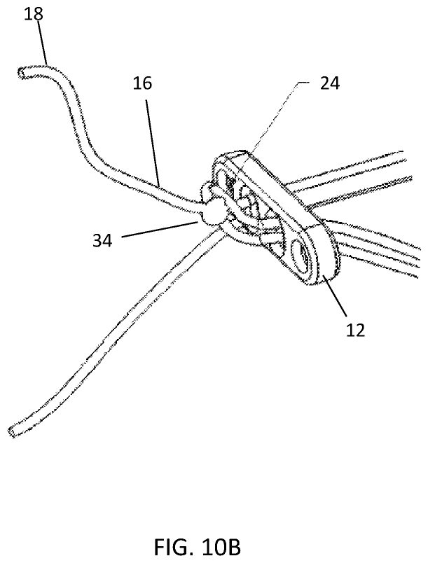

Turning now to FIG. 10A, there is shown a perspective view of the fixation device 10 connected to the tendon 38 in a second deployment configuration, according to an alternative embodiment. Once the adjustable loop 24 is passed through the button 12 and is on a proximal side of the button 12, the adjustable loop 24 is passed over the first terminal end 18 of the first limb 16 and over the protuberance 34. As shown in FIG. 10B, the adjustable loop 24 is positioned under the protuberance 34. The passing strand 28 (FIG. 9) can be removed from the fixation device 10 and the pre-sutured tendon 38 before or after the adjustable loop 24 is passed over the protuberance 34.

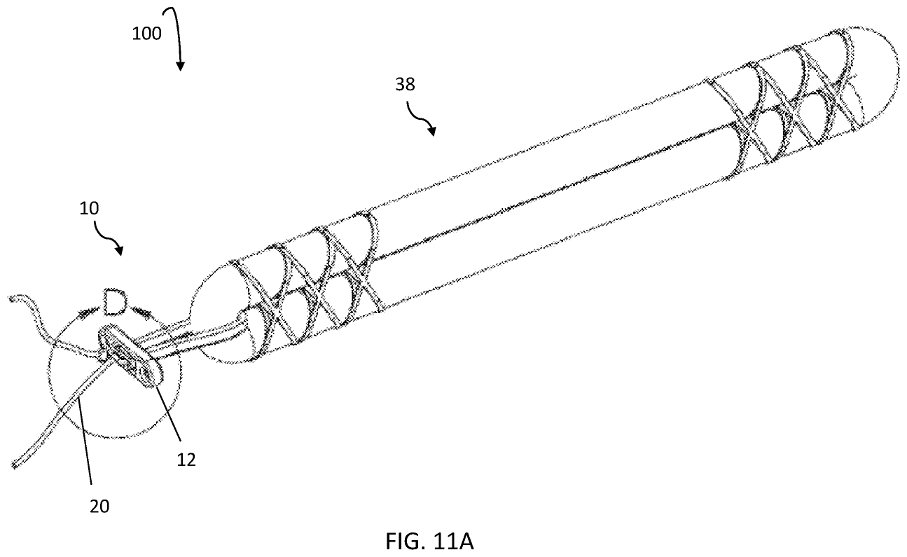

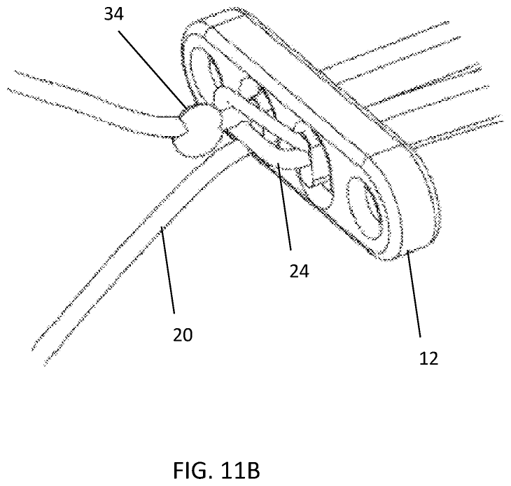

Referring now to FIG. 11A, there is shown a perspective view of the fixation device 10 connected to the tendon 38 in a post-deployment configuration, according to an alternative embodiment. The construct 100 is reduced by pulling the second limb 20. In particular, pulling the second limb 20 reduces the size (i.e., diameter or circumference) of the adjustable loop 24, bringing the pre-sutured tendon 38 toward the button 12. The second limb 20 is pulled until the desired length of the adjustable loop 24 is achieved and the pre-sutured tendon 38 is in the desired location respective to the button 12. Pulling the second limb 20 also captures the adjustable loop 24 underneath the protuberance 34, as shown in FIG. 11B. The resultant tension ensures the adjustable loop 24 cannot pass over the protuberance 34 and disengage.

Once the fixation device 10 is attached to the pre-sutured tendon 38, forming the fixation construct 100, the fixation construct 100 is passed through into a bone socket which communicated through both the inner and outer cortex. The fixation construct 100 is pulled through the bone socket using the first and second limbs 16, 20 until it emerges on the far cortex and flips on the surface. Details of the graft passing procedure are provided in U.S. Pat. No. 9,931,197, assigned to the assignee hereof and incorporated in its entirety herein by reference.

It should be understood that the values used above are only representative values, and other values may be in keeping with the spirit and intention of this disclosure.

While several inventive embodiments have been described and illustrated herein with reference to certain exemplary embodiments, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein (and it will be understood by one skilled in the art that various changes in detail may be effected therein without departing from the spirit and scope of the invention as defined by claims that can be supported by the written description and drawings). More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Further, where exemplary embodiments are described with reference to a certain number of elements it will be understood that the exemplary embodiments can be practiced utilizing either less than or more than the certain number of elements.

All references, including publications, patent applications, and patents, cited herein are hereby incorporated by reference to the same extent as if each reference were individually and specifically indicated to be incorporated by reference and were set forth in its entirety herein.

All definitions, as defined and used herein, should be understood to control over dictionary definitions, definitions in documents incorporated by reference, and/or ordinary meanings of the defined terms.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if not directly attached to where there is something intervening.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

The recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A fixation device, comprising:
a button having first and second inner apertures extending therethrough;
a length of suture having a first limb connected to a second limb, the first and second limbs both extending through the first inner aperture, forming an adjustable loop in the length of suture extending from the button;
a mass of suture material or a protuberance formed in a first terminal end of the first limb extending proximally from the button;
wherein the mass of suture material and the protuberance have a diameter that is larger than a diameter of the first inner aperture; and
wherein pulling the second limb proximally from the button decreases the size of the adjustable loop.

2. The device of claim 1, further comprising an eye-splice formed in first limb, the second limb passing through the eye-splice and the eye-splice extending distally from the button.

3. The device of claim 1, further comprising a pair of outer apertures extending through the button, wherein the first and second inner apertures are between the pair of outer apertures.

4. The device of claim 1, wherein the protuberance is a knot in the first limb.

5. The device of claim 1, wherein the mass of suture material is formed by passing a second terminal end of the second limb through the first limb at a plurality of passing locations.

6. The device of claim 1, further comprising a collapsible barb connected to the adjustable loop.

7. The device of claim 6, wherein the collapsible barb is composed of a hollow, braided tubular structure.

8. The device of claim 6, wherein the collapsible barb is a flat, woven structure passed through at multiple passing locations by the length of suture comprising the adjustable loop.

9. A fixation construct, comprising
a button having first and second inner apertures extending therethrough;
a length of suture having a first limb connected to a second limb, the first and second limbs both extending through the first inner aperture, forming an adjustable loop in the length of suture extending from the button;
a mass of suture material or a protuberance formed in a first terminal end of the first limb extending proximally from the button;
wherein the mass of suture material and the protuberance have a diameter that is larger than a diameter of the first inner aperture;
wherein pulling the second limb proximally from the button decreases the size of the adjustable loop; and
a tendon connected to the button by the adjustable loop such that the adjustable loop extends through the tendon and the second inner aperture and is secured on a proximal side of the button.

10. The construct of claim 9, wherein the tendon is a pre-sutured tendon.

11. The construct of claim 9, further comprising an eye-splice formed in first limb, the second limb passing through the eye-splice and the eye-splice extending distally from the button.

12. The construct of claim 9, further comprising a collapsible barb connected to the adjustable loop, wherein the collapsible barb is positioned over the first inner aperture on the proximal side of the button.

13. The construct of claim 9, wherein the adjustable loop is secured around and underneath the protuberance on the proximal side of the button.

14. A method of creating a fixation construct, comprising the steps of:
providing a fixation device having a button having first and second inner apertures extending therethrough, a length of suture having a first limb connected to a second limb, the first and second limbs both extending through the first inner aperture, forming an adjustable loop in the length of suture extending from the button, a mass of suture material or a protuberance formed in a first terminal end of the first limb extending proximally from the button, wherein the mass of suture material and the protuberance have a diameter that is larger than a diameter of the first inner aperture;
attaching a passing strand to the adjustable loop, the passing strand having a free end;
passing the free end of the passing strand through a tendon;
extending the free end of the passing strand through the second inner aperture until the adjustable loop is passed through the second inner aperture and extends from a proximal side of the button;
securing the adjustable loop on the proximal side of the button; and
pulling the second limb proximally from the button.

15. The method of claim 14, further comprising the step of removing the passing strand from the adjustable loop.

16. The method of claim 14, wherein the step of pulling the second limb proximally from the button decreases the size of the adjustable loop and pulls the tendon toward the button.

17. The method of claim 14, wherein the step of securing the adjustable loop on the proximal side of the button includes passing the adjustable loop over the first terminal end of the first limb and securing the adjustable loop around and underneath the protuberance on the proximal side of the button.

18. The method of claim 14, further comprising a collapsible barb connected to the adjustable loop.

19. The method of claim 18, wherein the step of securing the adjustable loop on the proximal side of the button includes tensioning the adjustable loop distally, causing the collapsible barb to expand radially over the second inner aperture.

20. The method of claim 18, further comprising an eye-splice formed in first limb, the second limb passing through the eye-splice and the eye-splice extending distally from the button.
